(19) United States
(12) Patent Application Publication
(10) Pub. No.: US 2004/0029122 A1
(43) Pub. Date: Feb. 12, 2004

(54) System and method for computer implemented glucose utilization defect risk assessment

(75) Inventors: Corinna Herrnstadt, San Diego, CA (US); Eoin D. Fahy, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: MitoKor, Inc., San Diego, CA

(21) Appl. No.: 10/218,191

(22) Filed: Aug. 12, 2002

(51) Int. Cl. C12Q 1/68; G06F 19/00; G01N 33/48; G01N 33/50

(52) U.S. Cl. 435/6; 702/20

(57) ABSTRACT

A computer implemented glucose utilization defect (GUD) risk assessment system and method screens individuals for GUD risk based upon their mitochondrial DNA sequence. A data input receives sample data, which is compared by a deviation determiner against the standard mitochondrial DNA reference to generate a binary output sequence containing difference positions indicating deviations between the sample data and the standard reference. A deviation analyzer then assesses GUD risk by analyzing the binary sequence output with respect to ranges of mitochondrial DNA positions as identified by pre-screen ranges stored in a pre-screen ranges database and GUD ranges stored in a GUD ranges database. The GUD ranges found in the GUD database are further determined by a reference string analysis method or a branch point analysis method performed by the GUD risk assessment system and method.

### BACKGROUND OF THE INVENTION

1. Field of the Invention

The invention relates generally to computer applications, systems and methods, and more particularly to computer systems and methods to assess risk for glucose utilization defects (GUD), such as type 2 diabetes mellitus in individuals.

2. Description of the Related Art

Glucose utilization defects (GUD) detrimentally affect individuals in many different ways. Unfortunately, glucose utilization defects have become widespread in large portions of populations in many areas of the world. Consequently, risk assessment for glucose utilization defects (GUD) in individuals has become an increasingly important field of endeavor. For an individual that may have a high risk for a glucose utilization defect, it can be vitally important for the individual to have advanced warning to assist in selecting proper lifestyle and treatment choices.

Examples of glucose utilization defects include type 2 diabetes mellitus, or “late onset” diabetes, which is a common, degenerative disease affecting 5 to 10 percent of the population in developed countries. The propensity for developing type 2 diabetes mellitus (“type 2 DM”) is reportedly maternally inherited, suggesting a mitochondrial genetic involvement. It has been found that diabetes has a strong genetic component; for instance, there is a high incidence of the disease among first degree relatives of affected individuals.

At the cellular level, the degenerative condition that may be characteristic of late onset diabetes mellitus includes indicators of altered mitochondrial respiratory function, for example, impaired insulin secretion, decreased ATP synthesis and increased levels of reactive oxygen species. Studies have shown that type 2 DM may be preceded by or associated with certain related disorders. For example, it is estimated that forty million individuals in the U.S. suffer from impaired glucose tolerance (IGT). Following a glucose load, circulating glucose concentrations in IGT patients rise to higher levels, and return to baseline levels more slowly, than in unaffected individuals. A small percentage of IGT individuals (5-10%) progress to non-insulin dependent diabetes (NIDDM) each year. This form of diabetes mellitus, type 2 DM, is associated with decreased release of insulin by pancreatic beta cells and a decreased end-organ response to insulin. Other symptoms of diabetes mellitus and conditions that precede or are associated with diabetes mellitus include obesity, vascular pathologies, peripheral and sensory neuropathies and blindness.

It is clear that none of the current pharmacological therapies corrects the underlying biochemical defects of glucose utilization defects such as in type 2 DM. Neither do any of these currently available treatments improve all of the physiological abnormalities in type 2 DM such as impaired insulin secretion, insulin resistance and/or excessive hepatic glucose output. In addition, treatment failures are common with these agents, such that multi-drug therapy is frequently necessary.

Due to the strong genetic component of diabetes mellitus, the nuclear genome has been the main focus of the search for causative genetic mutations. However, despite intense effort in searching, few nuclear genes associated with diabetes mellitus have been found. Those found include, for example, mutations in the insulin gene, the insulin receptor gene and the glucokinase gene. By comparison, a number of altered mitochondrial genes associated with diabetes mellitus have been reported. Unfortunately, relationships amongst mitochondrial and extramitochondrial factors that contribute to cellular respiratory and/or metabolic activities as they pertain to diabetes remain poorly understood.

Clearly there is a need for improved diagnostic methods for early detection in individuals at risk for developing glucose utilization defects, such as type 2 DM. The present invention provides computer based systems and methods directed toward such an end and offers other related advantages.

### BRIEF DESCRIPTION OF THE DRAWINGS

FIG. 1 is a schematic diagram of a computing system suitable for employing aspects of the present invention.

FIG. 2 is a schematic diagram illustrating a glucose utilization defect risk assessment system.

FIG. 3 is a flowchart illustrating a method performed by a deviation analyzer of the glucose utilization defect risk assessment system as shown in FIG. 2.

FIG. 4 is a flowchart illustrating a reference string analysis method used to generate glucose utilization defect ranges used by the glucose utilization defect risk assessment system as shown in FIG. 2.

FIG. 5 is a flowchart illustrating a branch point analysis method used as an alternative embodiment to generate glucose utilization defect ranges used by the glucose utilization defect risk assessment system as shown in FIG. 2.

### DETAILED DESCRIPTION OF THE INVENTION

A system and method for computer implemented glucose utilization defect risk assessment is described herein as the present invention. Embodiments of the present invention are used to assess risk for glucose utilization defects in individuals based upon computer-based analysis of mitochondrial DNA samples of the individuals. The present invention incorporates discoveries made by its originator that the mitochondrial DNA of special groups of individuals can be used to screen these individuals regarding risk for glucose utilization defects. These individuals are specially grouped according to their mitochondrial DNA, which is associated with certain special haplogroups selected from a group of conventionally defined haplogroups.

Mitochondrial DNA passes from a mother to her children, without any contribution from the father, resulting in the mitochondrial DNA staying intact without variation over many generations. The mutation rate for mitochondrial DNA has been found to be generally very slow, so that it can be used for studies such as those associated with genealogy. The conventionally defined haplogroups are identified based upon certain observed variations in the mitochondrial DNA compared with a standard mitochondrial DNA reference. Consequently, mitochondrial DNA of an individual can be compared with the standard mitochondrial DNA reference to determine which haplogroup is associated with the particular mitochondrial DNA of the individual.

For those individuals that have mitochondrial DNA that classifies them as being in one of certain special haplogroups, the present invention is able to screen these individuals regarding risk for glucose utilization defects by analyzing certain DNA segments of their mitochondrial DNA as compared to the standard mitochondrial DNA reference. The present invention generally uses a standard mitochondrial DNA reference known as the Cambridge Reference (Andrews et al. 1999, Nature Genetics, 23:147). When the mitochondrial DNA of an individual of one of the special haplogroups is compared with the Cambridge Reference Series, if a variation is found within at least one segment of a group of particularly selected segments, then the individual is classified as having risk for a glucose utilization defect. If no variations exist within at least one segment of the group of particularly selected segments of the mitochondrial DNA, then the individual is classified as not having significant risk for a glucose utilization defect.

In the following description, numerous specific details are provided to understand embodiments of the invention. One skilled in the relevant art, however, will recognize that the invention can be practiced without one or more of these specific details, or with other equivalent elements and components, etc. In other instances, well-known components and elements are not shown, or not described in detail, to avoid obscuring aspects of the invention or for brevity. In other instances, the invention may still be practiced if steps of the various methods described could be combined, added to, removed, or rearranged.

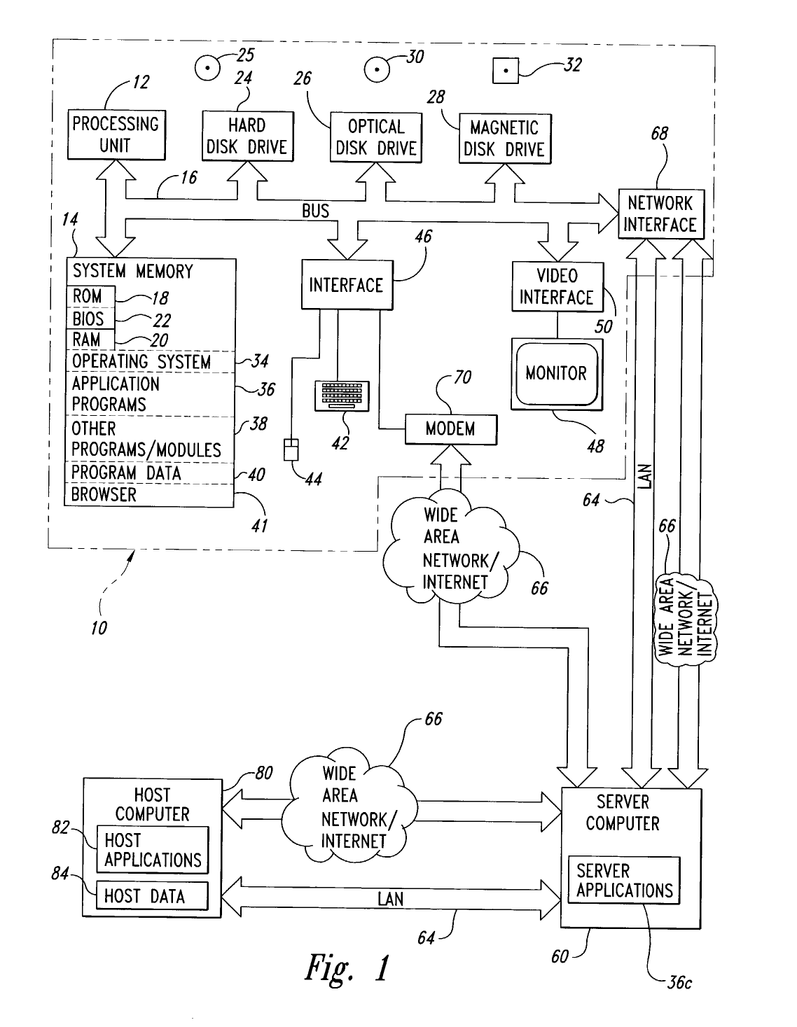

FIG. 1 and the following discussion provide a brief, general description of a suitable computing environment in which embodiments of the present invention can be implemented. Although not required, embodiments of the present invention will be described in the general context of computer-executable instructions, such as program application modules, objects, or macros being executed by a personal computer. Those skilled in the relevant art will appreciate that the invention can be practiced with other computer system configurations, including hand-held devices, multiprocessor systems, microprocessor-based or programmable consumer electronics, network PCs, mini computers, mainframe computers, and the like. The invention can be practiced in distributed computing environments where tasks or modules are performed by remote processing devices, which are linked through a communications network including wired and wireless environments. In a distributed computing environment, program modules may be located in both local and remote memory storage devices.

Referring to FIG. 1, a conventional personal computer, referred to herein as a client computer 10, includes a processing unit 12, a system memory 14 and a system bus 16 that couples various system components including the system memory to the processing unit. The client computer 10 will at times be referred to in the singular herein, but this is not intended to limited the application of the invention to a single client computer since in typical embodiments, there will be more than one client computer or other device involved. The processing unit 12 may be any logic processing unit, such as one or more central processing units (CPUs), digital signal processors (DSPs), application specific integrated circuits (ASICs), etc. Unless described otherwise, the construction and operation of the various blocks shown in FIG. 1 are of conventional design. As a result, such blocks need not be described in further detail herein, as they will be understood by those skilled in the relevant art.

The system bus 16 can employ any known bus structures or architectures, including a memory bus with memory controller, a peripheral bus, and a local bus. The system memory 14 includes read-only memory (“ROM”) 18 and random access memory (“RAM”) 20. A basic input/output system (“BIOS”) 22, which can form part of the ROM 18, contains basic routines that help transfer information between elements within the client computer 10, such as during start-up.

The client computer 10 also includes a hard disk drive 24 for reading from and writing to a hard disk 25, and an optical disk drive 26 and a magnetic disk drive 28 for reading from and writing to removable optical disks 30 and magnetic disks 32, respectively. The optical disk 30 can be a CD-ROM, while the magnetic disk 32 can be a magnetic floppy disk or diskette. The hard disk drive 24, optical disk drive 26 and magnetic disk drive 28 communicate with the processing unit 12 via the bus 16. The hard disk drive 24, optical disk drive 26 and magnetic disk drive 28 may include interfaces or controllers (not shown) coupled between such drives and the bus 16, as is known by those skilled in the relevant art. The drives 24, 26 and 28, and their associated computer-readable media, provide nonvolatile storage of computer readable instructions, data structures, program modules and other data for the client computer 10. Although the depicted client computer 10 employs hard disk 25, optical disk 30 and magnetic disk 32, those skilled in the relevant art will appreciate that other types of computer-readable media that can store data accessible by a computer may be employed, such as magnetic cassettes, flash memory cards, digital video disks (“DVD”), Bernoulli cartridges, RAMs, ROMs, smart cards, etc.

Program modules can be stored in the system memory 14, such as an operating system 34, one or more application programs 36, other programs or modules 38 and program data 40. The system memory 14 also includes a browser 41 for permitting the client computer 10 to access and exchange data with sources such as web sites of the Internet, corporate intranets, or other networks as described below, as well as other server applications on server computers such as those further discussed below. The browser 41 in the depicted embodiment is markup language based, such as Hypertext Markup Language (HTML), Extensible Markup Language (XML) or Wireless Markup Language (WML), and operates with markup languages that use syntactically delimited characters added to the data of a document to represent the structure of the document. Although the depicted embodiment shows the client computer 10 as a personal computer, in other embodiments, the client computer is some other computer related device such as a personal data assistant (PDA) or a cell phone or other mobile device.

While shown in FIG. 1 as being stored in the system memory 14, the operating system 34, application programs 36, other programs/modules 38, program data 40 and browser 41 can be stored on the hard disk 25 of the hard disk drive 24, the optical disk 30 of the optical disk drive 26 and/or the magnetic disk 32 of the magnetic disk drive 28. A user can enter commands and information into the client computer 10 through input devices such as a keyboard 42 and a pointing device such as a mouse 44. Other input devices can include a microphone, joystick, game pad, scanner, etc. These and other input devices are connected to the processing unit 12 through an interface 46 such as a serial port interface that couples to the bus 16, although other interfaces such as a parallel port, a game port or a wireless interface or a universal serial bus (“USB”) can be used. A monitor 48 or other display device is coupled to the bus 16 via a video interface 50, such as a video adapter. The client computer 10 can include other output devices, such as speakers, printers, etc.

The client computer 10 can operate in a networked environment using logical connections to one or more remote computers, such as a server computer 60. The server computer 60 can be another personal computer, a server, another type of computer, or a collection of more than one computer communicatively linked together and typically includes many or all of the elements described above for the client computer 10. The server computer 60 is logically connected to one or more of the client computers 10 under any known method of permitting computers to communicate, such as through a local area network (“LAN”) 64, or a wide area network (“WAN”) or the Internet 66. Such networking environments are well known in wired and wireless enterprise-wide computer networks, intranets, extranets, and the Internet. Other embodiments include other types of communication networks including telecommunications networks, cellular networks, paging networks, and other mobile networks.

When used in a LAN networking environment, the client computer 10 is connected to the LAN 64 through an adapter or network interface 68 (communicatively linked to the bus 16). When used in a WAN networking environment, the client computer 10 often includes a modem 70 or other device, such as the network interface 68, for establishing communications over the WAN/Internet 66. The modem 70 is shown in FIG. 1 as communicatively linked between the interface 46 and the WAN/Internet 66. In a networked environment, program modules, application programs, or data, or portions thereof, can be stored in the server computer 60. In the depicted embodiment, the client computer 10 is communicatively linked to the server computer 60 through the LAN 64 or the WAN/Internet 66 with TCP/IP middle layer network protocols; however, other similar network protocol layers are used in other embodiments. Those skilled in the relevant art will readily recognize that the network connections shown in FIG. 1 are only some examples of establishing communication links between computers, and other links may be used, including wireless links.

In some embodiments, the server computer 60 is further communicatively linked to a legacy host data system 80 typically through the LAN 64 or the WAN/Internet 66 or other networking configuration such as a direct asynchronous connection (not shown). With other embodiments, the client computer 10 is further communicatively linked (not shown) to the legacy host data system 80 typically through the LAN 64 or the WAN/Internet 66 or other networking configuration such as a direct asynchronous connection. Other embodiments may support the server computer 60 and the legacy host data system 80 by one computer system by operating all server applications and legacy host data system on the one computer system. The legacy host data system 80 in an exemplary embodiment is an International Business Machines (IBM) 390 mainframe computer configured to support IBM 3270 type terminals. Other exemplary embodiments use other vintage host computers such as IBM AS/400 series computers, UNISYS Corporation host computers, Digital Equipment Corporation VAX host computers and VT/Asynchronous host computers as the legacy host data system 80. The legacy host data system 80 is configured to run host applications 82 such as in system memory and store host data 84 such as business related data.

An exemplary embodiment of the invention is implemented in the Sun Microsystems Java programming language to take advantage of, among other things, the cross-platform capabilities found with the Java language. For instance, exemplary embodiments include the server computer 60 running Windows NT, Win2000, Solaris, Apple MacIntosh OS (e.g. 9.x or X) or Linux operating systems. In exemplary embodiments, the server computer 60 runs Apache Tomcat/Tomcat Jakarta web server or Microsoft Internet Information Server (ISS) web server, or BEA Weblogic web server.

Apache is a freely available Web server that is distributed under an “open source” license and runs on most UNIX-based operating systems (such as Linux, Solaris, Digital UNIX, and AIX), on other UNIX/POSIX-derived systems (such as Rhapsody, BeOs, and BS2000/OSD), on AmigaOS, and on Windows 2000/NT/95/98/ME. Windows-based systems with Web servers from companies such as Microsoft and Netscape are alternatives, but Apache web server seems suited for enterprises and server locations (such as universities) where UNIX-based systems are prevalent. Other embodiments use other web servers and programming languages such as C, C++, and C#.

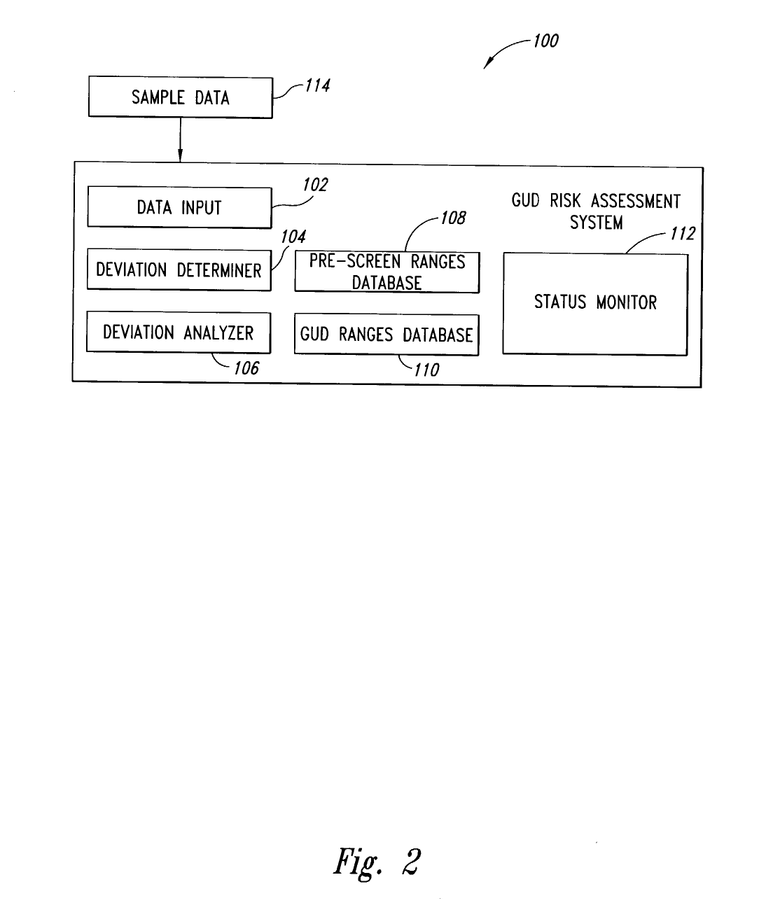

An embodiment of the present invention is illustrated as a GUD risk assessment system 100 as shown in FIG. 2. The GUD risk assessment system 100 can operate on one or more of the server computers 60 communicatively linked either by the LAN 64 or the WAN/Internet 66 to the client computer 10. In alternative embodiments, the GUD risk assessment system 100 operates on one of the client computers 10. The GUD risk assessment system 100 includes a data input 102, a deviation determiner 104, a deviation analyzer 106, a pre-screen database 108, a GUD ranges database 110, and a status monitor 112.

The data input 102 of the GUD risk assessment system 100 receives sample data 114 in the form of sample mitochondrial DNA sequences of individuals. The deviation determiner 104 compares the sample data 114 of an individual with the standard mitochondrial DNA reference, the Cambridge Reference Sequence (CRS, Andrews et al., 1999, Nature Genetics, 23:147) for the depicted embodiment, to determine differences between the sample data and the standard mitochondrial DNA reference and to identify the positions along the mitochondrial DNA sequence of the sample data in which the differences occur (referred to herein as difference positions). The CRS was first published in 1981 (Anderson, Nature, 290:457). The depicted embodiment, uses an updated version of the CRS published in 1999 (Andrews et al.), however, it is envisioned that further updates to the CRS could also be used by the present invention. The deviation determiner 104 further generates a binary sequence output of the sample data 114. The binary sequence output has ones in positions corresponding to the difference positions associated with the sample data 114 and zeroes elsewhere.

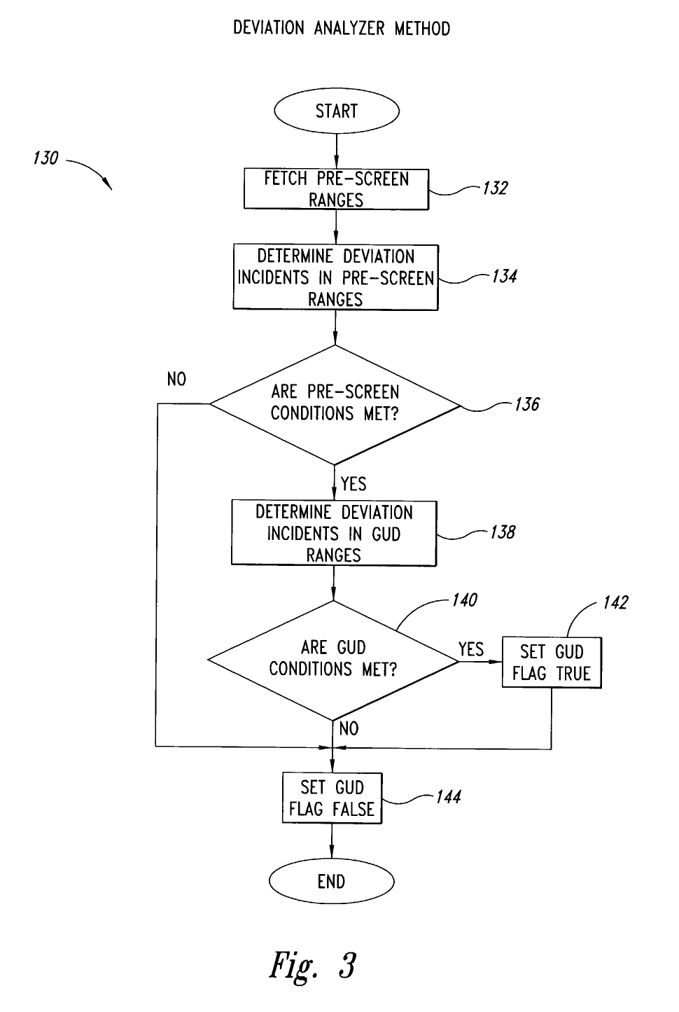

The deviation analyzer 106 then assesses GUD risk of the individual by analyzing the sample data 114 with respect to the special haplogroups identification stored in the pre-screen database 108 and analyzing the binary sequence output with respect to the GUD ranges stored in the GUD ranges database 110. Following a deviation analyzer method 130 shown in FIG. 3, the deviation analyzer 106 first fetches the special haplogroups identification (step 132) from the pre-screen database 108. The deviation analyzer 106 then compares the sample data 114 with the special haplogroups identification to determine if the sample data 114 falls into one of the special haplogroups, as discussed above, that are associated with potential GUD risk. Since the special haplogroups are particularly selected haplogroups, their identifications as haplogroups are conventionally known. In the depicted embodiment, the special haplogroups include the conventionally known haplogroups designated as LI, L2, L3, and H (see, for instance, Haplogroup Motifs http://www.stats.ox.ac.uk/˜macaulay/founder2000/motif.html, Mitochondrial DNA http://www.users.qwest.net/˜howery/mtdna.htm, Phylogenetic Analysis of Mitochondrial DNA http://herkules.oulu.fi/isbn9514255674/html/x367.html, and Chen Y S et al., Analysis of mtDNA Variation in African Populations Reveals the Most Agent of All Human Continent-Specific Haplogroups, Department of Genetics and Molecular Medicine, Emory University School of Medicine, Atlanta, Ga. 30322, USA, Chen et al., 2000 Am. J. Hum. Genet. 66: 1362-1383; Alves-Silva et al., 2000 Am. J Hum. Genet. 67:444-461; Wallace et al., 1999 Gene 238:211-230; Rando et al., 1998 Am. J Hu. Genet. 62:531-550; Watson et al., 1997 Am. J. Hum. Genet. 61:691-704; Chen et al., 1995 Am. J. Hum. Genet. 57:133-149). If the sample data 114 matches one of the special haplogroups identification, then the deviation analyzer method 130 branches (yes condition of decision step 136) to determine whether any difference positions of the binary output sequence generated from the sample data 114 fall within the GUD ranges (step 138) stored on the GUD ranges database 110. Otherwise (no condition of decision step 136), the deviation analyzer method 130 branches to set a GUD flag as false (step 144). If any of the difference positions of the binary sequence output fall within one of the GUD ranges associated with the sample data 114, the deviation analyzer method 130 branches (yes condition of decision step 140) to set the GUD flag as true. Otherwise (no condition of decision step 140), the GUD flag is set as false (step 144) and the deviation analyzer method 130 ends. The status monitor 112 of the GUD risk assessment system 100 displays results according to how the GUD flag is set by the deviation analyzer method 130. When the GUD flag is set to true, the status monitor 112 indicates that the individual has GUD risk whereas when the GUD flag is set to false the status monitor indicates that the test subject does not have significant GUD risk.

In a depicted embodiment, particular conventional mitochondrial loci, have defined ranges of DNA sequence positions, are used for the GUD ranges found in the GUD ranges database 110. It has been found that some of the special haplogroups may have different associated GUD ranges. For instance, in the depicted embodiment, for the haplogroups L1, L2, and L3, the following conventional loci (http://www.gen.emory.edu/mitomap.html) are used for the GUD ranges: RR2 (mitochondrial DNA sequence position numbers 1671-3229), CO2 (7586-8269), AT6 (8527-9207), CO3 (9207-9990), and ND3 (10059-10404). If the deviation analyzer 106 determines that the particular sample data 114 falls in either the L1, L2, or L3 haplogroups, then if the associated binary output sequence has a difference position in any of the positions of any of the RR2, CO2, AT6, CO3, and ND3 conventional loci listed above, then the individual associated with the sample data 114 is deemed to have risk of a glucose utilization defect.

Similarly, the H haplogroup, being a special haplogroup, has the following conventional loci, associated with GUD ranges, as follows: RR1 (648-1601), AT8 (8366-8572), ND4L (10470-10766), and ND5 (12337-14148). The GUD ranges for the H haplogroup also includes the following mitochondrial DNA sequence positions that come from one or more TR loci: 577-647, 1602-1670, 3230-3304, 4263-4331, 4329-4400, 4402-4469, 5512-5576, 5587-5655, 5657-5729, 5761-5826, 5826-5891, 7445-7516, 7518-7585, 8295-8364, 9991-10058, 10405-10469, 12138-12206, 12207-12265, 12266-12336, 14674-14742, 15888-15953, and 15955-16023. If the sample data 114 is associated with an H haplogroup test subject and the related binary output sequence has a difference position in any of the mitochondrial DNA sequence positions in at least one or more of the RR1, AT8, ND4L, or ND5 loci listed above, then the test subject is also deemed to have risk for a glucose utilization defect.

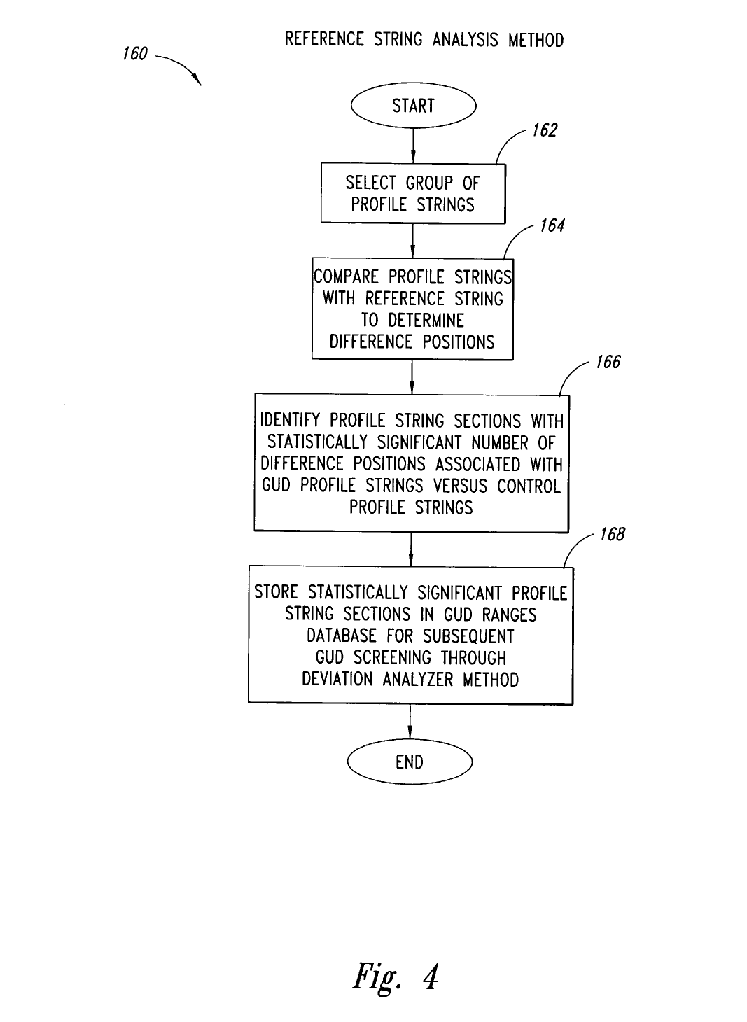

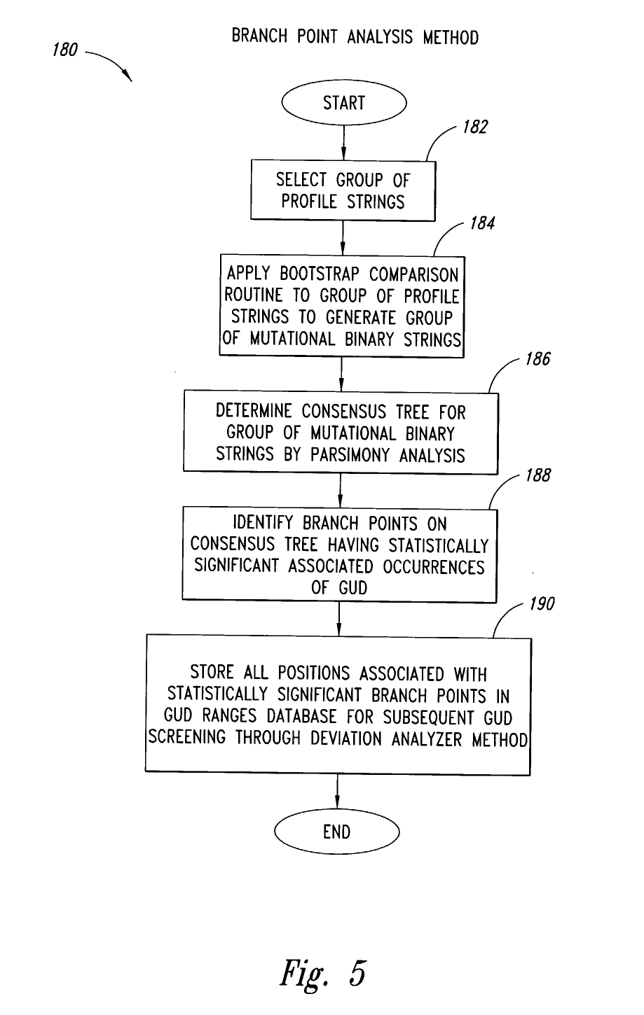

To determine GUD ranges for the various special haplogroups, the depicted embodiment can use either a reference string analysis method 160, as shown in FIG. 4, or a branch point analysis method 180, as shown in FIG. 5. The reference string analysis method 160 starts by selecting a group of profile character strings (step 162) in which some represent the mitochondrial DNA of individuals that have a glucose utilization defect and some represent those individuals that do not. The profile character strings are then compared with a reference character string, such as one associated with the CRS, to determine positions along the profile character strings (step 164) in which the profile character strings differ from the CRS to identify those positions as difference positions. The reference string analysis method 160 then identifies profile string sections which have a statistically significant number of difference positions associated with test subjects having a glucose utilization defect (step 166). These statistically significant profile string sections are then stored as GUD ranges in the GUD ranges database 110 (step 168) for subsequent GUD screening through the deviation analyzer method 130.

The branch point analysis method 180 starts by selecting a group of profile character strings (step 182) in which some represent the mitochondrial DNA of individuals that have a glucose utilization defect and some represent those individuals that do not. These profile character strings are then compared with each other in a bootstrap fashion to generate a group of mutational binary strings (step 184). For each profile character string, a mutational binary string is generated that identifies the sequence positions of the particular profile character string that differs from a consensus of the group of profile character strings. For each profile character string, a corresponding mutational binary string contains ones in the sequence positions of particular profile character strings where there is a deviation from the consensus of the group of profile character strings. Zeroes are contained elsewhere.

Conventional parsimony analysis is then used to determine a consensus tree for the group of mutational binary strings (step 186). In particular, the depicted embodiment uses a MIX program from a package of PHYLIP programs maintained and available from Dr. Joe Felsenstein of the Department of Genetics at the University of Washington (Seattle, Wash.). The MIX program estimates phylogenies by some parsimony methods for discrete character data with two states (0 and 1). The MIX program allows use of the Wagner parsimony method, the Camin-Sokal parsimony method, or arbitrary mixtures of these two parsimony methods. Other embodiments use other parsimony methods directed to discrete characters. PHYLIP (the PHYLogeny Inference Package) is a package of programs for inferring evolutionary or consensus trees. PHYLIP has been widely distributed since 1980 with over 6,000 registered users.

The branch point analysis method 180 then identifies branch points on the developed consensus tree that are associated with statistically significant occurrences of GUD (step 188) in which the deviations from the consensus of the group are associated with an individual having a GUD condition. The branch point analysis method 180 then stores all positions associated with statistically significant branch points in the GUD ranges data base 110 (step 190) for subsequent GUD screening through the deviation analyzer method 130.

From the foregoing it will be appreciated that, although specific embodiments of the invention have been described herein for purposes of illustration, various modifications may be made without deviating from the spirit and scope of the invention. Accordingly, the invention is not limited except as by the appended claims.

It is claimed:

1. A computer automated glucose utilization defect assessment system comprising:
a data input to receive sample data associated with mitochrondrial DNA of an individual;
a deviation determiner configured to generate a binary output sequence from the sample data with respect to a standard mitochondrial DNA reference, the binary output sequence having first positions containing a first binary value representing mitochondrial DNA sequence positions where differences exist in the first positions between the sample data and the standard mitochondrial DNA reference, the binary output sequence having second positions containing a second binary value representing mitochondrial DNA sequence positions where agreement exists in the second positions between the sample data and the standard mitochondrial DNA reference;
a pre-screen database containing identification associated with at least one special group of mitochondrial DNA;
a glucose utilization defect ranges database containing ranges of mitochondrial DNA positions associated with a glucose utilization defect condition; and
a deviation analyzer configured to assess risk of a glucose utilization defect condition for the individual based upon whether the sample data is associated with a special group of mitochondrial DNA sequences and whether at least one first position is in one of the glucose utilization defect ranges.

2. The system of claim 1 wherein the standard mitochondrial DNA reference is the Cambridge Reference.

3. The system of claim 1 wherein the special groups of mitochondrial DNA are special haplogroups.

4. The system of claim 3 wherein the special haplogroups include L1, L2, L3, and H.

5. The system of claim 4 wherein the utilization defect ranges associated with the special haplogroups L1, L2, and L3 include mitochondrial RR2, CO2, AT6, CO3, and ND3 loci.

6. The system of claim 4 wherein the utilization defect ranges associated with a special haplogroup H include mitochondrial RR1, AT8, ND4L, ND5 and TR loci.

7. A computer automated glucose utilization defect assessment system comprising:
a data input to receive sample data associated with mitochrondrial DNA of an individual;
a pre-screen database containing identification associated with at least one special group of mitochondrial DNA;
a glucose utilization defect ranges database containing ranges of mitochondrial DNA positions associated with a glucose utilization defect condition; and
a deviation analyzer configured to assess risk of a glucose utilization defect condition for the individual based upon whether the sample data is associated with identification of the pre-screen database and whether at least one first position of the sample data differs from a standard mitochondrial DNA reference in one of the glucose utilization defect ranges.

8. The system of claim 7 wherein the special groups of mitochondrial DNA are special haplogroups.

9. The system of claim 8 wherein the special haplogroups include L1, L2, L3, and H.

10. The system of claim 9 wherein the utilization defect ranges associated with the special haplogroups L1, L2, and L3 include mitochondrial RR2, CO2, AT6, CO3, and ND3 loci.

11. The system of claim 9 wherein the utilization defect ranges associated with a special haplogroup H include mitochondrial RR1, AT8, ND4L, ND5 and TR loci.

12. A computer automated glucose utilization defect assessment system comprising:
a data input to receive sample data associated with mitochrondrial DNA of an individual;
a deviation determiner configured to generate a binary output sequence from the sample data with respect to a standard mitochondrial DNA reference, the binary output sequence having first positions containing a first binary value representing mitochondrial DNA sequence positions where differences exist in the first positions between the sample data and the standard mitochondrial DNA reference, the binary output sequence having second positions containing a second binary value representing mitochondrial DNA sequence positions where agreement exists in the second positions between the sample data and the standard mitochondrial DNA reference;
a glucose utilization defect ranges database containing ranges of mitochondrial DNA positions associated with a glucose utilization defect condition; and
a deviation analyzer configured to assess risk of a glucose utilization defect condition for the individual based upon whether at least one first position is in one of the glucose utilization defect ranges.

13. The system of claim 12 wherein the standard mitochondrial DNA reference is the Cambridge Reference.

14. A computer automated glucose utilization defect assessment system comprising:
a data input to receive sample data associated with mitochrondrial DNA of an individual;
a glucose utilization defect ranges database containing ranges of mitochondrial DNA positions associated with a glucose utilization defect condition; and
a deviation analyzer configured to assess risk of a glucose utilization defect condition for the individual based upon whether the sample data differs from a standard mitochondrial DNA reference in at least one mitochondrial position of one of the glucose utilization defect ranges.

15. A computer-implemented method comprising:
receiving sample data associated with mitochrondrial DNA of an individual;
generating a binary output sequence from the sample data with respect to a standard mitochondrial DNA reference, the binary output sequence having first positions containing a first binary value representing mitochondrial DNA sequence positions where differences exist in the first positions between the sample data and the standard mitochondrial DNA reference, the binary output sequence having second positions containing a second binary value representing mitochondrial DNA sequence positions where agreement exists in the second positions between the sample data and the standard mitochondrial DNA reference;
storing identification associated with at least one special group of mitochondrial DNA;
storing ranges of mitochondrial DNA positions associated with a glucose utilization defect condition; and
assessing risk of a glucose utilization defect condition for the individual based upon whether the sample data is associated with a special group of mitochondrial DNA sequences and whether at least one first position is in one of the glucose utilization defect ranges.

16. The method of claims 15 wherein the standard mitochondrial DNA reference is the Cambridge Reference.

17. The method of claims 15 wherein the special groups of mitochondrial DNA are special haplogroups.

18. The method of claim 17 wherein the special haplogroups include L1, L2, L3, and H.

19. The method of claim 18 wherein the utilization defect ranges associated with the special haplogroups L1, L2, and L3 include mitochondrial RR2, CO2, AT6, CO3, and ND3 loci.

20. The method of claim 18 wherein the utilization defect ranges associated with a special haplogroup H include mitochondrial RR1, AT8, ND4L, ND5 and TR loci.

21. A computer-implemented method comprising:
receiving sample data associated with mitochrondrial DNA of an individual;
storing identification associated with at least one special group of mitochondrial DNA;
storing ranges of mitochondrial DNA positions associated with a glucose utilization defect condition; and
assessing risk of a glucose utilization defect condition for the individual based upon whether the sample data is associated with identification of the pre-screen database and whether at least one first position of the sample data differs from a standard mitochondrial DNA reference in one of the glucose utilization defect ranges.

22. The method of claim 21 wherein the special groups of mitochondrial DNA are special haplogroups.

23. The method of claim 22 wherein the special haplogroups include L1, L2, L3, and H.

24. The method of claim 23 wherein the utilization defect ranges associated with the special haplogroups L1, L2, and L3 include mitochondrial RR2, CO2, AT6, CO3, and ND3 loci.

25. The method of claim 23 wherein the utilization defect ranges associated with a special haplogroup H include mitochondrial RR1, AT8, ND4L, ND5 and TR loci.

26. A computer-implemented method comprising:
receiving sample data associated with mitochrondrial DNA of an individual;
generating a binary output sequence from the sample data with respect to a standard mitochondrial DNA reference, the binary output sequence having first positions containing a first binary value representing mitochondrial DNA sequence positions where differences exist in the first positions between the sample data and the standard mitochondrial DNA reference, the binary output sequence having second positions containing a second binary value representing mitochondrial DNA sequence positions where agreement exists in the second positions between the sample data and the standard mitochondrial DNA reference;
storing ranges of mitochondrial DNA positions associated with a glucose utilization defect condition; and
assessing risk of a glucose utilization defect condition for the individual based upon whether at least one first position is in one of the glucose utilization defect ranges.

27. The method of claim 26 wherein the standard mitochondrial DNA reference is the Cambridge Reference.

28. A computer-implemented method comprising:
receiving sample data associated with mitochrondrial DNA of an individual;
storing ranges of mitochondrial DNA positions associated with a glucose utilization defect condition; and
assessing risk of a glucose utilization defect condition for the individual based upon whether the sample data differs from a standard mitochondrial DNA reference in at least one mitochondrial position of one of the glucose utilization defect ranges.

29. A computer-readable medium whose contents cause a computer to perform assessment of glucose utilization defects by:
receiving sample data associated with mitochrondrial DNA of an individual;
generating a binary output sequence from the sample data with respect to a standard mitochondrial DNA reference, the binary output sequence having first positions containing a first binary value representing mitochondrial DNA sequence positions where differences exist in the first positions between the sample data and the standard mitochondrial DNA reference, the binary output sequence having second positions containing a second binary value representing mitochondrial DNA sequence positions where agreement exists in the second positions between the sample data and the standard mitochondrial DNA reference;
storing identification associated with at least one special group of mitochondrial DNA;
storing ranges of mitochondrial DNA positions associated with a glucose utilization defect condition; and
assessing risk of a glucose utilization defect condition for the individual based upon whether the sample data is associated with a special group of mitochondrial DNA sequences and whether at least one first position is in one of the glucose utilization defect ranges.

30. The system of claim 29 wherein the standard mitochondrial DNA reference is the Cambridge Reference.

31. The system of claim 29 wherein the special groups of mitochondrial DNA are special haplogroups.

32. The system of claim 31 wherein the special haplogroups include L1, L2, L3, and H.

33. The system of claim 32 wherein the utilization defect ranges associated with the special haplogroups L1, L2, and L3 include mitochondrial RR2, CO2, AT6, CO3, and ND3 loci.

34. The system of claim 32 wherein the utilization defect ranges associated with a special haplogroup H include mitochondrial RR1, AT8, ND4L, ND5 and TR loci.

35. A computer-readable medium whose contents cause a computer to perform assessment of glucose utilization defects by:
receiving sample data associated with mitochrondrial DNA of an individual;
storing identification associated with at least one special group of mitochondrial DNA;
storing ranges of mitochondrial DNA positions associated with a glucose utilization defect condition; and
assessing risk of a glucose utilization defect condition for the individual based upon whether the sample data is associated with identification of the pre-screen database and whether at least one first position of the sample data differs from a standard mitochondrial DNA reference in one of the glucose utilization defect ranges.

36. The system of claim 35 wherein the special groups of mitochondrial DNA are special haplogroups.

37. The system of claim 36 wherein the special haplogroups include L1, L2, L3, and H.

38. The system of claim 37 wherein the utilization defect ranges associated with the special haplogroups L1, L2, and L3 include mitochondrial RR2, CO2, AT6, CO3, and ND3 loci.

39. The system of claim 37 wherein the utilization defect ranges associated with a special haplogroup H include mitochondrial RR1, AT8, ND4L, ND5 and TR loci.

40. A computer-readable medium whose contents cause a computer to perform assessment of glucose utilization defects by:
receiving sample data associated with mitochrondrial DNA of an individual;
generating a binary output sequence from the sample data with respect to a standard mitochondrial DNA reference, the binary output sequence having first positions containing a first binary value representing mitochondrial DNA sequence positions where differences exist in the first positions between the sample data and the standard mitochondrial DNA reference, the binary output sequence having second positions containing a second binary value representing mitochondrial DNA sequence positions where agreement exists in the second positions between the sample data and the standard mitochondrial DNA reference;
storing ranges of mitochondrial DNA positions associated with a glucose utilization defect condition; and
assessing risk of a glucose utilization defect condition for the individual based upon whether at least one first position is in one of the glucose utilization defect ranges.

41. The system of claim 40 wherein the standard mitochondrial DNA reference is the Cambridge Reference.

42. A computer-readable medium whose contents cause a computer to perform assessment of glucose utilization defects by:
receiving sample data associated with mitochrondrial DNA of an individual;
storing ranges of mitochondrial DNA positions associated with a glucose utilization defect condition; and
assessing risk of a glucose utilization defect condition for the individual based upon whether the sample data differs from a standard mitochondrial DNA reference in at least one mitochondrial position of one of the glucose utilization defect ranges.
